(19) United States
(12) Patent Application Publication
(10) Pub. No.: US 2010/0260851 A1
(43) Pub. Date: Oct. 14, 2010

(54) Novel Polymorph of Atorvastatin Calcium and Use Thereof for the Preparation of Amorphous Atorvastatin Calcium

(75) Inventors: Girish Dixit, Uttar Pradesh (IN); Anil Shahaji Khile, Navi Mumbai (IN); Nitin Sharadchandra Pradhan, Thane (West) (Maharashtra) (IN); Jon Valgeirsson, Hafnarfjordur (IS)

Correspondence Address: CANTOR COLBURN, LLP, 20 Church Street, 22nd Floor, Hartford, CT, 06103, US

(73) Assignee: ACTAVIS GROUP PTC EHF, Hafnarfjordur (IS)

(21) Appl. No.: 12/668,433

(22) PCT Filed: Jul. 11, 2008
(86) PCT No.: PCT/IB08/02624

§ 371 Date: May 10, 2010

(30) Foreign Application Priority Data
Jul. 11, 2007 (IN) 1494/CHE/2007
Jul. 30, 2007 (IN) 1649/CHE/2007
Aug. 3, 2007 (IN) 1710/CHE/2007

(51) Int. Cl. A61K 9/14; A61K 31/40; C07D 207/34; A61P 3/06; A61P 9/10

(52) U.S. Cl. 424/489; 514/423; 548/537

(57) ABSTRACT

The present invention provides a novel polymorphic form of atorvastatin calcium, designated as form Al, process for preparation, pharmaceutical compositions, and method of treating thereof. The present invention further provides a process for the preparation of highly pure amorphous atorvastatin calcium using the novel atorvastatin calcium form Al. The present invention also relates to novel amorphous form of atorvastatin tert-butyl ester, chemically known as [R-(R*,R*)]-2-(4-fluorophenyl)-[β],[δ]-dihydroxy -5-(1-methylethyl)-3-phenyl-4-(phenylcarbamoyl-1H-pyrrole-1-heptanoicacid tert-butyl ester, process for the preparation, and its application for preparing highly pure atorvastatin and its pharmaceutically acceptable salts thereof. The present invention also relates to use of the novel amorphous atorvastatin tert-butyl ester and novel atorvastatin calcium form al for preparing amorphous atorvastatin calcium.

### CROSS REFERENCE TO RELATED APPLICATION

This application claims the benefit of priority to Indian provisional application Nos. 1494/CHE/2007, filed on Jul. 11, 2007, 1649/CHE/2007, filed on Jul. 30, 2007 and 1710/CHE/2007, filed on Aug. 3, 2007, which are incorporated herein by reference.

### FIELD OF THE INVENTION

The present invention provides a novel polymorphic form of atorvastatin calcium, designated as Form A1, process for preparation, pharmaceutical compositions, and method of treating thereof. The present invention further provides a process for the preparation of highly pure amorphous atorvastatin calcium using the novel atorvastatin calcium Form A1. The present invention also relates to novel amorphous form of atorvastatin tert-butyl ester, chemically known as [R—(R*,R*)]-2-(4-fluorophenyl)-[β], [δ]-dihydroxy-5-(1-methylethyl)-3-phenyl-4-(phenylcarbarnoyl)-1H-pyrrole-1-heptanoicacid tert-butyl ester, process for the preparation, and its application for preparing highly pure atorvastatin and its pharmaceutically acceptable salts thereof. The present invention also relates to use of the novel amorphous atorvastatin tert-butyl ester and novel atorvastatin calcium Form A1 for preparing amorphous atorvastatin calcium.

### BACKGROUND OF THE INVENTION

Atorvastatin, chemically known as [R—(R*,R*)]-2-(4-fluorophenyl)-β,δ-dihydroxy-5-(1-methylethyl)-3-phenyl-4-[(phenyl amino)carbonyl]-1H-pyrrole-1-heptanoic acid, is an important reductase inhibitor of the enzyme 3-hydroxy-3-methylglutarate-coenzyme A (HMG-CoA) and therefore is a useful anti-hyperlipoproteinemic agent. It has proven to be a highly effective medicament for the treatment of disorders such as hyperlipidemia and hypercholesterolemia which are conditions that are known risk factors for arteriosclerosis and coronary heart disease. Atorvastatin is represented by the following structural formula I:

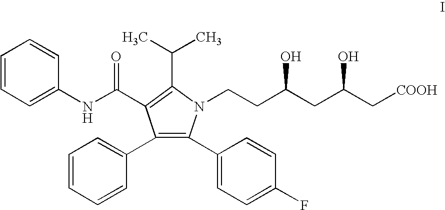

and is marketed as the hemi calcium salt-trihydrate under the name LIPITOR by Warner-Lambert Co. Atorvastatin calcium is used as a lipid-lowering agent for the treatment of hypercholesterolemia and represented by the following structural formula II:

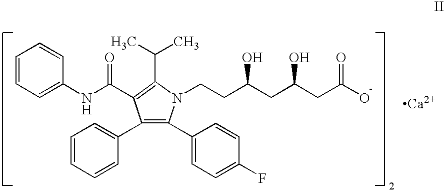

U.S. Pat. No. 5,929,156 discloses crystalline Form I of atorvastatin calcium hydrate, oral formulations comprising it, crystalline Form II atorvastatin calcium and hydrates thereof and crystalline Form IV atorvastatin calcium and hydrates thereof.

U.S. Pat. No. 6,121,461 discloses crystalline Form III of atorvastatin calcium hydrate, which is also useful as hypolipidemic and hypocholesterolemic agent.

PCT Patent Publication No. WO 01/36384 discloses the Form V of atorvastatin calcium and hydrates thereof, process for preparation and pharmaceutical compositions containing thereof.

PCT Patent Publication No. WO 03/011826, U.S. Patent Application No. 2004/0242899 and U.S. Pat. No. 7,074,818 disclose crystalline forms VI and VII of atorvastatin calcium and processes for preparing these forms.

The calcium salt of atorvastatin enables atorvastatin to be conveniently formulated in, for example, tablets, capsules, lozenges, powders, and the like for oral administration. Additionally, there is a need to produce atorvastatin in a pure and crystalline form to enable formulations meeting stability, dissolution and bioavailability requirements.

Atorvastatin tert-butyl ester, chemically [R—(R*,R*)]-2-(4-fluorophenyl)-[β],[δ]-dihydroxy-5-(1-methylethyl)-3-phenyl-4-(phenylcarbamoyl)-1H-pyrrole-1-heptanoic acid tert-butyl ester, of formula III:

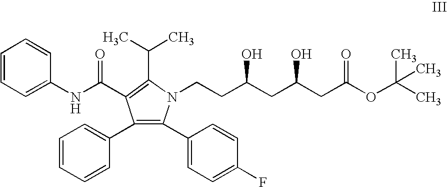

is a known and valuable precursor for the preparation of the HMG-CoA reductase inhibitor atorvastatin and pharmaceutically acceptable salts thereof.

Tetrahedron Lett., 1992, 33, 2283-2284 discloses the preparation of dimethyl ketal of atorvastatin tert-butyl ester, chemically (4R-cis)-6-[2-[3-phenyl-4-(phenylcarbamoyl)-2-(4-fluorophenyl)-5-(1-methylethyl)-pyrrol-1-yl]-ethyl]-2,2-dimethyl-[1,3]-dioxane-4-yl-acetic acid tert-butyl ester, of formula IV:

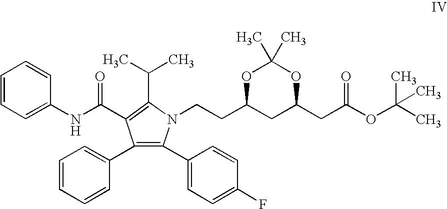

by a Paal-Knorr pyrrole synthesis using a ternary solvent mixture of toluene-heptane-tetrahydrofuran (1:4:1) with catalysis by pivalic acid and conversion of (IV) to atorvastatin hemi-calcium without isolating any intermediates.

PCT Publication No. WO 02/059087 describes the direct conversion of the dimethyl ketal of atorvastatin tert-butyl ester of formula IV into non-crystalline atorvastatin hemi-calcium or atorvastatin lactone without isolating intermediary occurring products.

PCT Publication Nos. WO 02/083637 and WO 02/083638 disclose that the treatment of dimethyl ketal of atorvastatin tert-butyl ester of formula IV in methanol with aqueous hydrochloric acid yielded a complicated mixture containing 5 intermediates, as revealed by HPLC analysis.

The percentage and distribution of the detected compounds in the reaction mixture varied depending on the reaction conditions. However, none of the intermediates, including atorvastatin tert-butyl ester of formula III, was isolated, but the reaction solution was further treated with dilute aqueous sodium hydroxide solution.

PCT Publication Nos. WO 02/43667 and WO 03/016317 also reported difficulties during the preparation of atorvastatin hemi-calcium, in particular during the step of converting dimethyl ketal of atorvastatin tert-butyl ester of formula IV using an acid catalyst to atorvastatin tert-butyl ester of formula III. The product proved to contain other compounds, such as atorvastatin lactone and atorvastatin free acid, and was isolated e.g. as an oil which indicates that atorvastatin tert-butyl ester of formula III prepared in this manner is not substantially pure. Thus, these processes were not able to provide atorvastatin tert-butyl ester of formula III in a well defined amorphous form of high purity.

PCT Patent Publication No. WO 2005/097742 Al discloses two crystalline forms (Forms I and II) of atorvastatin tert-butyl ester of formula III, and characterizes them by powder X-ray diffraction (P-XRD) and Differential Scanning Calorimetry (DSC).

Therefore, the processes of the prior art are not capable of producing atorvastatin tert-butyl ester of formula III in amorphous form and therefore a form of high purity.

The preparation of atorvastatin hemi calcium salt in the above mentioned processes suffers with low purity of the intermediates i.e. atorvastatin tert-butyl ester, which is especially problematic since the desired form of the finished product is amorphous atorvastatin hemi calcium. In addition, current processes are not optimal in terms of production capabilities and are not suitable for large scale manufacturing.

Hence there is need to provide an improved process for the manufacture of atorvastatin hemi-calcium relative to the prior art processes without the problems associated therewith.

Polymorphism is defined as “the ability of a substance to exist as two or more crystalline phases that have different arrangement and/or conformations of the molecule in the crystal lattice. Thus, in the strict sense, polymorphs are different crystalline forms of the same pure substance in which the molecules have different arrangements and/or configurations of the molecules”. Different polymorphs may differ in their physical properties such as melting point, solubility, X-ray diffraction patterns, etc. Although those differences disappear once the compound is dissolved, they can appreciably influence pharmaceutically relevant properties of the solid form, such as handling properties, dissolution rate and stability. Such properties can significantly influence the processing, shelf life, and commercial acceptance of a polymorph. It is therefore important to investigate all solid forms of a drug, including all polymorphic forms, and to determine the stability, dissolution and flow properties of each polymorphic form. Polymorphic forms of a compound can be distinguished in the laboratory by analytical methods such as X-ray diffraction (XRD), Differential Scanning Calorimetry (DSC) and infrared spectrometry (IR).

Solvent medium and mode of isolation play very important role in obtaining a polymorphic form over the other.

It has been disclosed in the art that the amorphous forms in a number of drugs exhibit different dissolution characteristics and in some cases different bioavailability patterns compared to crystalline forms [Konne T., Chem. Pharm. Bull., 38, 2003-2007 (1990)]. For some therapeutic indications one bioavailability pattern may be favored over another.

The discovery of new polymorphic forms of a pharmaceutically useful compound provides a new opportunity to improve the performance characteristics of a pharmaceutical product. It enlarges the repertoire of materials that a formulation scientist has available for designing, for example, a pharmaceutical dosage form of a drug with a targeted release profile or other desired characteristic.

Accordingly, there remains a need in the art for a novel, stable and substantially pure polymorphic form of atorvastatin calcium. Since the novel polymorphic form of atorvastatin calcium, designated as Form A1, obtained with high purity, the said From A1 can be used to obtain amorphous atorvastatin calcium.

There is also a continuing need for improved methods for preparation of amorphous atorvastatin calcium.

### SUMMARY OF THE INVENTION

The present inventors have now surprisingly and unexpectedly discovered a novel polymorphic form of atorvastatin calcium and a novel amorphous form of atorvastatin tert-butyl ester with high purity, adequate stability and good dissolution properties. In one aspect, the present invention provides a novel and stable polymorphic form of atorvastatin calcium, designated as atorvastatin calcium polymorphic Form A1, characterized by an X-ray powder diffraction pattern having peaks expressed as 2-theta angle positions at about 5.3, 8.3 and 15.7±0.2 degrees.

In another aspect, the present invention further encompasses a process for preparing the highly pure and stable polymorphic form A1 of atorvastatin calcium.

In another aspect, the present invention further encompasses use of the novel atorvastatin calcium polymorphic Form A1 for the preparation of amorphous atorvastatin calcium.

In another aspect, the present invention further provides a process for preparing amorphous atorvastatin calcium with high purity by using the highly pure and stable atorvastatin calcium polymorphic Form A1 of the present invention.

In another aspect, the present invention provides a novel and stable amorphous form of atorvastatin tert-butyl ester and use thereof for the preparation of atorvastatin or a pharmaceutically acceptable salt thereof, preferably atorvastatin calcium, and most preferably amorphous atorvastatin calcium.

In another aspect, the present invention further encompasses a process for preparing the highly pure and stable amorphous form of atorvastatin tert-butyl ester.

In another aspect, the present invention provides a pharmaceutical composition comprising atorvastatin calcium polymorphic Form A1 of the present invention and one or more pharmaceutically acceptable excipients.

In still another aspect, the present invention provides a pharmaceutical composition comprising atorvastatin calcium polymorphic Form A1 made by the process of the present invention, and one or more pharmaceutically acceptable excipients.

In still further aspect, the present invention further encompasses a process for preparing a pharmaceutical formulation comprising combining atorvastatin calcium polymorphic Form A1 with one or more pharmaceutically acceptable excipients.

In another aspect, the atorvastatin calcium polymorphic Form A1 disclosed herein for use in the pharmaceutical compositions of the present invention, wherein 90 volume-percent of the particles (D₉₀) have a size of less than or equal to about 500 microns, specifically less than or equal to about 300 microns, more specifically less than or equal to about 200 microns, still more specifically less than or equal to about 100 microns, and most specifically less than or equal to about 15 microns.

In another aspect, the present invention provides atorvastatin calcium polymorphic Form A1 having relatively low content of one or more organic volatile impurities.

Unless otherwise indicated, the following definitions are set forth to illustrate and define the meaning and scope of the various terms used to describe the invention herein.

The term “polymorphic form” refers to a crystal modification that can be characterized by analytical methods such as X-ray powder diffraction, IR-spectroscopy, differential scanning calorimetry (DSC) or by its melting point.

The term “amorphous” means a solid without long-range crystalline order. Amorphous form of amorphous atorvastatin tert-butyl ester in accordance with the present invention preferably contains less than about 10% crystalline forms of atorvastatin tert-butyl ester, more preferably less than 5% crystalline forms of atorvastatin tert-butyl ester, and still more preferably is essentially free of crystalline forms of atorvastatin tert-butyl ester. Similarly, Amorphous form of amorphous atorvastatin calcium in accordance with the present invention preferably contains less than about 10% crystalline forms of atorvastatin calcium, more preferably less than 5% crystalline forms of atorvastatin calcium, and still more preferably is essentially free of crystalline forms of atorvastatin calcium. “Essentially free of crystalline forms of atorvastatin tert-butyl ester or atorvastatin calcium” means that no crystalline polymorph forms of atorvastatin tert-butyl ester or atorvastatin calcium can be detected within the limits of a powder X-ray diffractometer.

The term “pharmaceutically acceptable” means that which is useful in preparing a pharmaceutical composition that is generally non-toxic and is not biologically undesirable and includes that which is acceptable for veterinary use and/or human pharmaceutical use.

The term “pharmaceutical composition” is intended to encompass a drug product including the active ingredient(s), pharmaceutically acceptable excipients that make up the carrier, as well as any product which results, directly or indirectly, from combination, complexation or aggregation of any two or more of the ingredients. Accordingly, the pharmaceutical compositions of the present invention encompass any composition made by admixing the active ingredient, active ingredient dispersion or composite, additional active ingredient(s), and pharmaceutically acceptable excipients.

The expression “pharmaceutically acceptable salt ” is meant those salts which are, within the scope of sound medical judgment, suitable for use in contact with the tissues of humans and lower animals without undue toxicity, irritation, allergic response and the like, commensurate with a reasonable benefit/risk ratio, and effective for their intended use. Representative alkali or alkaline earth metal salts include the sodium, calcium, potassium and magnesium salts, and the like.

The term “therapeutically effective amount” as used herein means the amount of a compound that, when administered to a mammal for treating a state, disorder or condition, is sufficient to effect such treatment. The “therapeutically effective amount” will vary depending on the compound, the disease and its severity and the age, weight, physical condition and responsiveness of the mammal to be treated.

The term “delivering” as used herein means providing a therapeutically effective amount of an active ingredient to a particular location within a host causing a therapeutically effective blood concentration of the active ingredient at the particular location. This can be accomplished, e.g., by topical, local or by systemic administration of the active ingredient to the host.

The term “buffering agent” as used herein is intended to mean a compound used to resist a change in pH upon dilution or addition of acid of alkali. Such compounds include, by way of example and without limitation, potassium metaphosphate, potassium phosphate, monobasic sodium acetate and sodium citrate anhydrous and dehydrate and other such material known to those of ordinary skill in the art.

The term “sweetening agent” as used herein is intended to mean a compound used to impart sweetness to a formulation. Such compounds include, by way of example and without limitation, aspartame, dextrose, glycerin, mannitol, saccharin sodium, sorbitol, sucrose, fructose and other such materials known to those of ordinary skill in the art.

The term “binders” as used herein is intended to mean substances used to cause adhesion of powder particles in granulations. Such compounds include, by way of example and without limitation, acacia alginic acid, tragacanth, carboxymethylcellulose sodium, polyvinylpyrrolidone, compressible sugar (e.g., NuTab), ethylcellulose, gelatin, liquid glucose, methylcellulose, povidone and pregelatinized starch, combinations thereof and other material known to those of ordinary skill in the art. If required, other binders may also be included in the present invention.

Exemplary binders include starch, polyethylene glycol, guar gum, polysaccharide, bentonites, sugars, invert sugars, poloxamers (PLURONIC™ F68, PLURONIC™ F127), collagen, albumin, celluloses in nonaqueous solvents, combinations thereof and the like. Other binders include, for example, polypropylene glycol, polyoxyethylene-polypropylene copolymer, polyethylene ester, polyethylene sorbitan ester, polyethylene oxide, microcrystalline cellulose, polyvinylpyrrolidone, combinations thereof and other such materials known to those of ordinary skill in the art.

The term “diluent” or “filler” as used herein is intended to mean inert substances used as fillers to create the desired bulk, flow properties, and compression characteristics in the preparation of solid dosage formulations. Such compounds include, by way of example and without limitation, dibasic calcium phosphate, kaolin, sucrose, mannitol, microcrystalline cellulose, powdered cellulose, precipitated calcium carbonate, sorbitol, starch, combinations thereof and other such materials known to those of ordinary skill in the art.

The term “glidant” as used herein is intended to mean agents used in solid dosage formulations to improve flow-properties during tablet compression and to produce an anti-caking effect. Such compounds include, by way of example and without limitation, colloidal silica, calcium silicate, magnesium silicate, silicon hydrogel, cornstarch, talc, combinations thereof and other such materials known to those of ordinary skill in the art.

The term “lubricant” as used herein is intended to mean substances used in solid dosage formulations to reduce friction during compression of the solid dosage. Such compounds include, by way of example and without limitation, calcium stearate, magnesium stearate, mineral oil, stearic acid, zinc stearate, combinations thereof and other such materials known to those of ordinary skill in the art.

The term “disintegrant” as used herein is intended to mean a compound used in solid dosage formulations to promote the disruption of the solid mass into smaller particles which are more readily dispersed or dissolved. Exemplary disintegrants include, by way of example and without limitation, starches such as corn starch, potato starch, pregelatinized, sweeteners, clays, such as bentonite, macrocrystalline cellulose (e.g. Avicel™), carsium (e.g. Amberlite™), alginates, sodium starch glycolate, gums such as agar, guar, locust bean, karaya, pectin, tragacanth, combinations thereof and other such materials known to those of ordinary skill in the art.

The term “wetting agent” as used herein is intended to mean a compound used to aid in attaining intimate contact between solid particles and liquids. Exemplary wetting agents include, by way of example and without limitation, gelatin, casein, lecithin (phosphatides), gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters, (e.g., TWEEN™s), polyethylene glycols, polyoxyethylene stearates colloidal silicon dioxide, phosphates, sodium dodecylsulfate, carboxymethylcellulose calcium, carboxymethylcellulose sodium, methylcellulose, hydroxyethylcellulose, hydroxyl propylcellulose, hydroxypropylmethylcellulose phthalate, noncrystalline cellulose, magnesium aluminum silicate, triethanolamine, polyvinyl alcohol, and polyvinylpyrrolidone (PVP). Tyloxapol (a nonionic liquid polymer of the alkyl aryl polyether alcohol type) is another useful wetting agent, combinations thereof and other such materials known to those of ordinary skill in the art.

As used herein, D_{X} means that X percent of the particles have a diameter less than a specified diameter D. Thus, a D₉₀ or d(0.9) of less than 300 microns means that 90 volume-percent of the micronized particles in a composition have a diameter less than 300 microns.

The term “micronization” used herein means a process or method by which the size of a population of particles is reduced.

As used herein, the term “micron” or “μm” both are same refers to “micrometer” which is 1×10⁻⁶ meter.

As used herein, “crystalline particles” means any combination of single crystals, aggregates and agglomerates.

As used herein, “Particle Size Distribution (P.S.D)” means the cumulative volume size distribution of equivalent spherical diameters as determined by laser diffraction in Malvern Master Sizer 2000 equipment or its equivalent. “Mean particle size distribution, i.e., D₅₀” correspondingly, means the median of said particle size distribution.

By “substantially pure” is meant having purity greater than about 99%, specifically greater than about 99.5%, and more specifically greater than about 99.9% measured by HPLC.

### BRIEF DESCRIPTION OF THE DRAWINGS

FIG. 1 is a characteristic powder X-ray diffraction (XRD) pattern of atorvastatin calcium polymorphic Form A1.

FIG. 2 is a characteristic infra red (IR) spectrum of atorvastatin calcium polymorphic Form A1.

FIG. 3 is a characteristic differential scanning calorimetric (DSC) thermogram of atorvastatin calcium polymorphic Form A1.

FIG. 4 is a characteristic powder X-ray diffraction (XRD) pattern of amorphous atorvastatin calcium.

FIG. 5 is a characteristic powder X-ray diffraction (XRD) pattern of amorphous atorvastatin tert-butyl ester.

The X-Ray powder diffraction was measured by an X-ray powder diffractometer equipped with a Cu-anode (?=1.54 Angstrom), X-ray source operated at 40 kV, 40 mA and a Ni filter is used to strip K-beta radiation. Two-theta calibration is performed using an NIST SRM 1976, Corundum standard. The sample was analyzed using the following instrument parameters: measuring range=3-45° 2?; step width=0.01579°; and measuring time per step=0.11 second.

Differential scanning calorimetric (DSC) thermogram of atorvastatin calcium Form A1 has an endotherm range at 164-167° C. This has been recorded on Jade DSC (Perkin-Elmer, USA) under the nitrogen gas purge at a flow of 20 mL/min. The instrument was calibrated for temperature and heat flow using indium as standard. Data acquisition and analysis were performed using pyres software. Atorvastatin calcium Form A1 may contain up to 4% water, which corresponds to the stoichiometric value of 9 water molecules per molecule of atorvastatin calcium. Thus, atorvastatin calcium Form A1 can be in various states of hydration, between 0 and 9 moles of water.

FT-IR spectroscopy was carried out with a Perkin Elmer Spectrum 100 series spectrometer. For the production of the KBr compacts approximately 2 mg of sample was powdered with 200 mg of KBr. The spectra were recorded in transmission mode ranging from 4000 to 450 cm⁻¹.

### DETAILED DESCRIPTION OF THE INVENTION

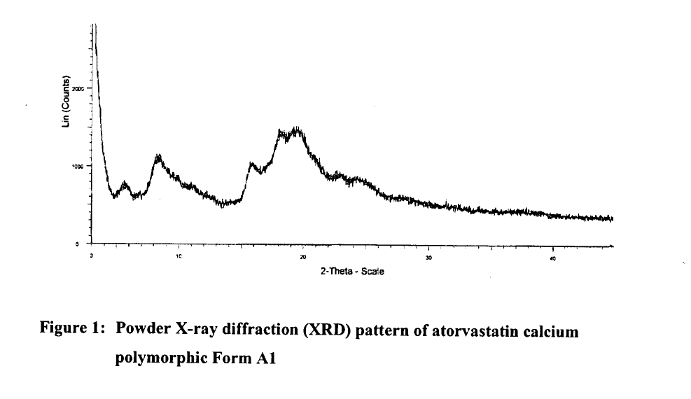

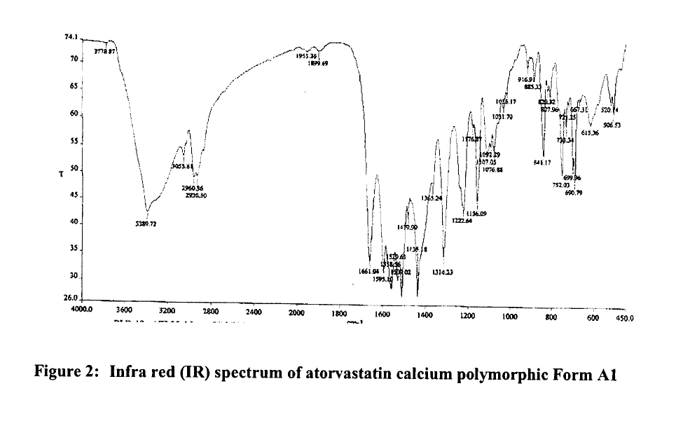

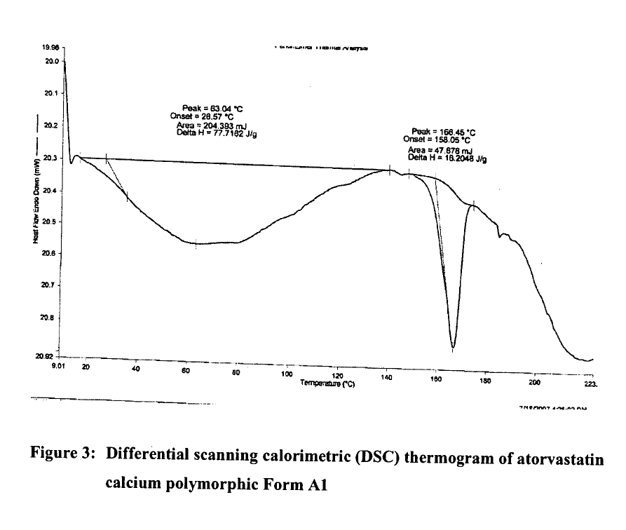

According to one aspect of the present invention, there is provided a novel polymorphic form of atorvastatin calcium, designated as polymorphic Form A1, characterized by at least one, and preferably all, of the following properties:
i) a powder X-ray diffraction pattern substantially in accordance with FIG. 1;
ii) a powder X-ray diffraction pattern having peaks at about 5.3, 8.3 and 15.7±0.2 degrees 2-theta substantially as depicted in FIG. 1;
iii) an IR spectrum substantially in accordance with FIG. 2;
iv) an IR spectrum having characteristic absorption bands at about 1661, 820 and 807 cm⁻¹; and
v) a DSC thermogram having an endotherm peak at about 164-167° C. substantially as depicted in FIG. 3.

The new polymorphic form of atorvastatin calcium Form A1 is well distinguished from the crystal modifications obtained by carrying out the procedures described in the prior art using X-ray powder diffraction, Infra red spectroscopy and Diffrential scanning calorimetry.

According to another aspect of the present invention, a process is provided for preparation of stable and substantially pure atorvastatin calcium polymorphic Form A1, which comprises:
a) providing a solution of an atorvastatin salt in water;
b) optionally, adjusting the pH of the solution with an acid;
c) optionally, seeding the solution obtained in step-(a) or step-(b) with amorphous atorvastatin calcium;
d) combining the atorvastatin salt solution with a suitable calcium salt; and
e) isolating polymorphic Form A1 of atorvastatin calcium from the reaction mass.

The process can produce polymorphic Form A1 of atorvastatin calcium in substantially pure form.

The term “substantially pure atorvastatin calcium polymorphic Form A1” refers to the atorvastatin calcium polymorphic Form A1 having purity greater than about 98%, specifically greater than about 99%, more specifically greater than about 99.5% and still more specifically greater than about 99.9% (measured by HPLC).

The atorvastatin calcium polymorphic Form A1 is stable, consistently reproducible and has good flow properties, and which is particularly suitable for bulk preparation and handling, and so, the novel atorvastatin calcium polymorphic Form A1 is suitable for formulating atorvastatin calcium. Moreover, the atorvastatin calcium polymorphic Form A1 is useful intermediate in the preparation of amorphous atorvastatin calcium in high purity.

The atorvastatin salt used in step-(a) includes atorvastatin alkali metal salts such as lithium, sodium and potassium salts; atorvastatin alkaline-earth metal salts such as magnesium, strontium salts; as well as ammonium and alkyl, aryl or alkaryl ammonium salts of atorvastatin. The preferred atorvastatin salts are alkali metal salts; and most preferably sodium salt.

Step-(a) of providing a solution of atorvastatin salt includes dissolving atorvastatin salt in water, or obtaining an existing solution from a previous processing step.

Preferably the atorvastatin salt is dissolved in water at a temperature of below about 90° C., more preferably at about 30° C. to about 80° C., and still more preferably at about 40° C. to about 70° C.

The solution of atorvastatin salt may also be prepared by hydrolyzing atorvastatin tert-butyl ester of formula III, preferably amorphous atorvastatin tert-butyl ester of the present invention, with a suitable base in a suitable solvent under suitable conditions to produce a reaction mass containing crude atorvastatin salt followed by usual work up such as washings, evaporations, extractions etc., and dissolving the resulting atorvastatin salt in water under stirring at a temperature of below about 90° C., more preferably at about 30° C. to about 80° C., and still more preferably at about 40° C. to about 70° C. The suitable base is an inorganic base.

Exemplary inorganic bases include, but are not limited to, ammonia; hydroxides, carbonates and bicarbonates of alkali or alkaline earth metals. Specific alkali metals are lithium, sodium and potassium, and more specifically sodium and potassium. Specific alkaline earth metals are magnesium and strontium, and more specifically magnesium.

Specific inorganic bases are ammonia, sodium hydroxide, magnesium hydroxide, potassium hydroxide, lithium hydroxide, sodium carbonate, potassium carbonate, lithium carbonate, and more specifically sodium hydroxide.

The solution obtained in step-(a) may optionally be subjected to carbon treatment. The carbon treatment can be carried out by methods known in the art, for example by stirring the solution with finely powdered carbon at a temperature of below about 70° C. for at least 15 minutes, preferably at a temperature of about 40° C. to about 70° C. for at least 30 minutes; and filtering the resulting mixture through hyflo to obtain a filtrate containing atorvastatin salt by removing charcoal. Preferably, finely powdered carbon is an active carbon.

The pH of the solution in step-(b) is optionally adjusted at about 7-9 and preferably at about 7.5-8.5 with an acid. The acid used to adjust the pH can be an organic or inorganic acid. In a preferred embodiment, the acid is an aqueous solution of an inorganic acid. Specific acids are hydrochloric acid, hydrobromic acid, sulfuric acid, nitric acid, acetic acid, propionic acid, phosphoric acid, succinic acid, maleic acid, fumaric acid, citric acid, glutaric acid, citraconic acid, glutaconic acid, tartaric acid, malic acid, ascorbic acid, and more specifically hydrochloric acid.

The calcium salt used in step-(d) includes organic and inorganic salts of calcium which are capable of dissociating into Ca²⁺ and an anionic component when added to the atorvastatin salt solution. Among the organic salts that may be used are carboxylates and sulfonates. Exemplary carboxylates are lower alkyl carboxylates such as acetate, proprionate, butyrate and tartrate; aryl carboxylates such as benzoate and phthalate; and higher alkyl carboxylates such as stearate, dodecanoate and the like. Also included are calcium ascorbate and succinate. Among the sulfonates that may be used are lower alkyl and aryl sulfonates like calcium methane sulfonate, calcium benzene sulfonate and calcium p-toluene sulfonate. The preferred organic calcium salts are lower carboxylate salts, and the most preferred organic calcium salt is calcium acetate.

Depending upon solubility, inorganic salts which may be used include halide salts such as CaCl₂, CaF₂, CaBr₂ and CaI₂, as well as calcium borate (B₄CaO₇), calcium tetrafluoroborate (CaBF₄), calcium carbonate (CaCO₃), monobasic calcium phosphate (Ca(H₂PO₄)₂), dibasic calcium phosphate (CaHPO₄) and tribasic calcium phosphate (Ca(PO₄)₂), calcium sulfate (CaSO₄) and calcium hydroxide (Ca(OH)₂), and hydrates thereof.

Whether organic or inorganic, the calcium salt is preferably added in an amount that provides one half mole of Ca²⁺ per mole of atorvastatin in the atorvastatin salt solution. For example, if the atorvastatin salt is atorvastatin sodium (atorvastatin⁻Na⁺), then about one half mole of calcium salt per mole of the atorvastatin salt is appropriate. If the atorvastatin salt is atorvastatin magnesium ([atorvastatin⁻]₂ Mg²⁺), then about one mole of calcium salt per mole of atorvastatin salt is appropriate. Otherwise, mixed salts containing atorvastatin may form.

The combining of the atorvastatin salt solution with a suitable calcium salt may be done in any order, for example, the calcium salt may be added to the atorvastatin salt solution, or alternatively, the atorvastatin salt solution may be added to the calcium salt. The addition may be carried out drop wise or in one portion or in more than one portion. The addition is preferably carried out slowly at a temperature of below about 50° C. for at least 15 minutes, and more preferably at a temperature of about 15° C. to about 35° C. from about 30 minutes to about 2 hours.

Preferably, the calcium salt may be combined with the atorvastatin salt solution by adding the calcium salt in substantially pure form, i.e. either as a solid or, if liquid, as a neat liquid, to the atorvastatin salt solution or, more preferably, by first forming a calcium salt solution and then combining the atorvastatin salt solution with calcium salt solution. It is most preferred to combine the calcium salt and the atorvastatin salt solution by first dissolving the calcium salt in a solvent and then adding the calcium salt solution to the atorvastatin salt solution slowly. The preferred calcium salt is calcium acetate and the preferred calcium salt solvent is water. When the calcium salt solvent is water, it is preferably used in an amount that provides about a 20 to 30 millimolar solution of the calcium salt, more preferably about a 25 millimolar solution.

After completion of addition process, the resulting mass is preferably stirred at a temperature of below about 50° C. for at least 30 minutes, and more preferably at a temperature of about 25° C. to about 50° C. from about 2 hours to 12 hours.

The isolation of pure atorvastatin calcium polymorphic Form A1 in step-(e) may be carried out by forcible or spontaneous crystallization.

Spontaneous crystallization refers to crystallization without the help of an external aid such as seeding, cooling etc., and forcible crystallization refers to crystallization with the help of an external aid.

Forcible crystallization may be initiated by a method usually known in the art such as cooling, seeding, partial removal of the solvent from the solution, by adding an anti-solvent to the solution, or a combination thereof.

The term “Anti-solvent” refers to a solvent which when added to an existing solution of a substance reduces the solubility of the substance.

Preferably the crystallization is carried out by cooling the solution at a temperature of below 30° C., and more preferably at about 0° C. to about 25° C. The solid obtained in step-(e) is collected by filtration or centrifugation.

The atorvastatin calcium polymorphic Form A1 obtained by above process may be further dried in, for example, Vacuum Tray Dryer, Rotocon Vacuum Dryer, Vacuum Paddle Dryer or pilot plant Rota vapor, to further lower residual solvents. Drying can be carried out under reduced pressure until the residual solvent content reduces to the desired amount such as an amount that is within the limits given by the International Conference on Harmonization of Technical Requirements for Registration of Pharmaceuticals for Human Use (“ICH”) guidelines.

The total purity of the atorvastatin calcium polymorphic Form A1 obtained by the process disclosed herein is of greater than about 99%, specifically greater than about 99.9%, and more specifically greater than about 99.99% as measured by HPLC.

Amorphous atorvastatin calcium can be prepared in high purity by using the substantially pure atorvastatin calcium polymorphic Form A1 of the present invention, by the methods disclosed hereinafter.

In one embodiment, the substantially pure atorvastatin calcium polymorphic Form A1 disclosed herein for use in the pharmaceutical compositions of the present invention, wherein 90 volume-percent of the particles (D₉₀) have a size of less than or equal to about 400 microns, specifically less than or equal to about 300 microns, more specifically less than or equal to about 200 microns, still more specifically less than or equal to about 100 microns, and most specifically less than or equal to about 15 microns.

In another embodiment, the particle sizes of substantially pure atorvastatin calcium polymorphic Form A1 can be achieved by a mechanical process of reducing the size of particles which includes any one or more of cutting, chipping, crushing, milling, grinding, micronizing, trituration or other particle size reduction methods known in the art, to bring the solid state forms the desired particle size range.

According to another aspect of the present invention, there is provided a method for treating or preventing diseases caused by disorders such as hyperlipidemia and hypercholesterolemia, comprising administering the atorvastatin calcium polymorphic Form A1, or a pharmaceutical composition that comprises atorvastatin calcium polymorphic Form A1, along with pharmaceutically acceptable excipients.

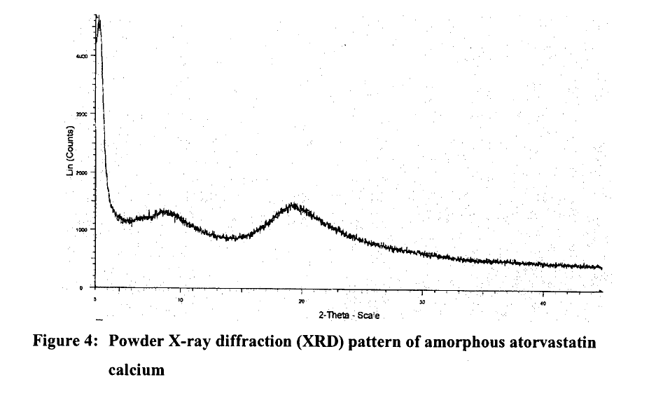

According to another aspect of the present invention, a process is provided for preparation of a stable and substantially pure amorphous form of atorvastatin calcium, characterized by a powder XRD pattern substantially in accordance with FIG. 4, which comprises:
a) providing a solution of atorvastatin calcium polymorphic Form A1 in a suitable solvent selected from the group comprising halogenated hydrocarbons, ketones, and mixtures thereof;
b) optionally, filtering the solvent solution to remove any extraneous matter; and
c) substantially removing the solvent from the solution to afford amorphous form of atorvastatin calcium.

The process can produce amorphous atorvastatin calcium in substantially pure form.

The term “substantially pure amorphous form of atorvastatin calcium” refers to the amorphous form of atorvastatin calcium having purity greater than about 98%, specifically greater than about 99%, more specifically greater than about 99.5% and still more specifically greater than about 99.9% (measured by HPLC).

The amorphous atorvastatin calcium obtained by the process disclosed herein is stable, consistently reproducible and has good flow properties, and which is particularly suitable for bulk preparation and handling, and so, the atorvastatin calcium obtained by the process disclosed herein is suitable for formulating atorvastatin calcium.

Preferable halogenated hydrocarbons are dichloromethane, 1,2-dichloroethane, chloroform, carbon tetrachloride, and mixtures thereof, and more preferably dichloromethane. Exemplary ketone solvents include, but are not limited to, acetone, methyl ethyl ketone, methyl isobutyl ketone, methyl tert-butyl ketone and the like, and mixtures thereof. Specific ketone solvent is acetone, methyl ethyl ketone or a mixture thereof.

Preferably, when the organic solvent is halogenated hydrocarbon then the ratio of atorvastatin calcium salt to the organic solvent is about 0.5 g/mL.

Preferably, when the organic solvent is ketone then the ratio of atorvastatin calcium salt to the organic solvent is about 26 to 39% w/w.

Step-(a) of providing a solution of atorvastatin calcium polymorphic Form A1 includes dissolving atorvastatin calcium polymorphic Form A1 in the solvent or by suspending the atorvastatin calcium polymorphic Form A1 in the solvent followed by heating the suspension to form a clear solution.

Preferably the dissolution is carried out at a temperature of below about boiling temperature of the solvent used, more preferably at about 25° C. to about 100° C., and still more preferably at about 40° C. to about 80° C.

The solution obtained in step-(a) may optionally be subjected to carbon treatment. The carbon treatment can be carried out by methods known in the art, for example by stirring the solution with finely powdered carbon at a temperature of below about 70° C. for at least 15 minutes, preferably at a temperature of about 40° C. to about 70° C. for at least 30 minutes; and filtering the resulting mixture through hyflo to obtain a filtrate containing atorvastatin calcium by removing charcoal. Preferably, finely powdered carbon is an active carbon.

The solution obtained in step-(a) or step-(b) is optionally stirred at a temperature of about 30° C. to the reflux temperature of the solvent used for at least 20 minutes, and preferably at a temperature of about 40° C. to the reflux temperature of the solvent used from about 30 minutes to about 4 hours.

Removal of solvent in step-(c) is accomplished by, for example, substantially complete evaporation of the solvent, concentrating the solution and filtering the solid under inert atmosphere. Alternatively, the solvent may also be removed by evaporation. Evaporation can be achieved at sub-zero temperatures by the lyophilization or freeze-drying technique. The solution may also be completely evaporated in, for example, a pilot plant Rota vapor, a Vacuum Paddle Dryer or in a conventional reactor under vacuum above about 720 mm Hg by flash evaporation techniques by using an agitated thin film dryer (“ATFD”), or evaporated by spray drying to obtain a dry amorphous powder.

One of the preferred methodologies to remove the solvent involves spray-drying, in which a solution of atorvastatin calcium is sprayed into the spray drier at the flow rate ranging from 10 to 300 ml/hr, preferably flow rate is 40 to 200ml/hr. The air inlet temperature to the spray drier used may range from 25 to 150° C., preferably from 60° C. to 110° C. and the outlet air temperature used may range from 30 to 90° C. Another preferred method is vertical agitated thin-film drying (or evaporation). Agitated thin film evaporation technology involves separating the volatile component using indirect heat transfer coupled with mechanical agitation of the flowing film under controlled condition.

The distillation process can be performed at atmospheric pressure or reduced pressure. Preferably the solvent is removed at a pressure of about 760 mm Hg or less, more preferably at about 400 mm Hg or less, still more preferably at about 80 mm Hg or less, and most preferably from about 30 to about 80 mm Hg.

The substantially pure amorphous form of atorvastatin calcium obtained by the above processes may be further dried in, for example, Vacuum Tray Dryer, Rotocon Vacuum Dryer, Vacuum Paddle Dryer or pilot plant Rota vapor, to further lower residual solvents.

The total purity of the amorphous atorvastatin calcium obtained by the process disclosed herein is of greater than about 99.9%, specifically greater than about 99.95%, and more specifically greater than about 99.99% as measured by HPLC.

According to another aspect of the present invention, there is provided a stable and substantially pure amorphous form of atorvastatin tert-butyl ester of formula III:

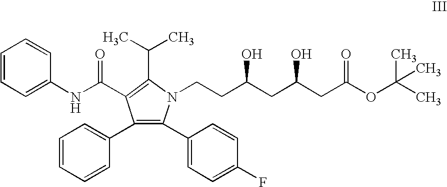

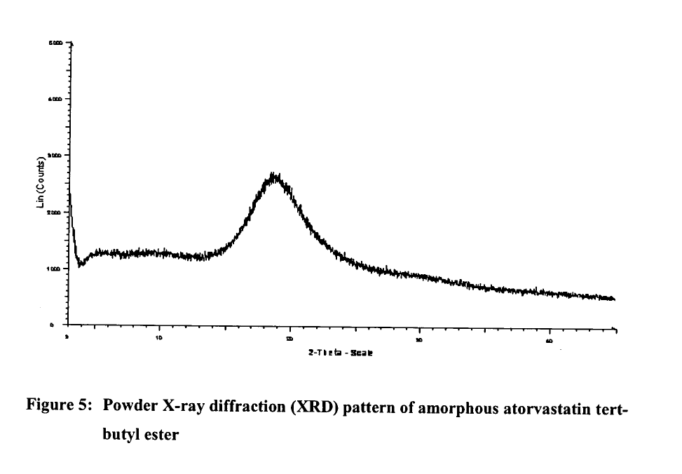

Amorphous form of atorvastatin tert-butyl ester is characterized by a powder XRD pattern substantially in accordance with FIG. 5. The X-ray powder diffraction pattern shows no peaks that are characteristic of amorphous form of atorvastatin tert-butyl ester, thus demonstrating the amorphous nature of the product.

According to another aspect of the present invention, a process is provided for preparation of a stable and substantially pure amorphous form of atorvastatin tert-butyl ester of formula III, which comprises:
a) providing a solution of atorvastatin tert-butyl ester in a suitable water immiscible solvent;
b) optionally, filtering the solvent solution to remove any extraneous matter; and
c) substantially removing the solvent from the solution to afford amorphous form of atorvastatin tert-butyl ester.

The process can produce amorphous atorvastatin tert-butyl ester in substantially pure form.

The term “substantially pure amorphous form of atorvastatin tert-butyl ester” refers to the amorphous form of atorvastatin tert-butyl ester having purity greater than about 98%, specifically greater than about 99%, more specifically greater than about 99.5% and still more specifically greater than about 99.9% (measured by HPLC).

The amorphous atorvastatin tert-butyl ester is stable, consistently reproducible and has good flow properties, and which is particularly suitable for bulk preparation. Moreover, the amorphous form of atorvastatin tert-butyl ester is useful intermediate in the preparation of atorvastatin and its pharmaceutically acceptable salts in high purity.

Exemplary water immiscible solvents used in step-(a) include, but are not limited to aromatic hydrocarbons, cyclic ethers, halogenated organic solvents and the like, and mixtures thereof. Preferable aromatic hydrocarbon solvents include, but are not limited to, toluene, xylene and the like and mixtures thereof. Preferable cyclic ether solvents include, but are not limited to, tetrahydrofuran, 1,4-dioxan, hexahydropyran and the like and mixtures thereof. Preferable halogenated organic solvents include, but are not limited to, dichloromethane, 1,2-dichloroethane, chloroform, carbon tetrachloride and the like and mixtures thereof. More preferable water immiscible solvent used in step-(a) is dichloromethane.

Step-(a) of providing a solution of atorvastatin tert-butyl ester includes dissolving atorvastatin tert-butyl ester in the water immiscible solvent or obtaining an existing solution containing the atorvastatin tert-butyl ester from a previous processing step followed by extraction with the water immiscible solvent.

Preferably the atorvastatin tert-butyl ester is dissolved in the suitable water immiscible solvent at a temperature of below about reflux temperature of the solvent used, more preferably at about 20° C. to about 110° C., and still more preferably at about 25° C. to about 80° C.

As used herein, “reflux temperature” means the temperature at which the solvent or solvent system refluxes or boils at atmospheric pressure.

The solution in step-(a) may also be prepared by condensing (4R-cis)-1,1-dimethylethyl 6-(2-aminoethyl)-2,2-dimethyl-1,3-dioxane-4-acetate with (f)-4-fluoro-α-(2-methyl-1-oxopropyl)-γ-oxo-N,β-diphenyl benzenebutaneamide under acidic conditions in a suitable solvent or a mixture of suitable solvents to produce (4R-cis)-6-[2-[3-phenyl-4-(phenylcarbamoyl)-2-(4-fluorophenyl)-5-(1-methylethyl)-pyrrol-1-yl]-ethyl]-2,2-dimethyl-[1,3]-dioxane-4-yl-acetic acid tert-butyl ester of formula IV, which is then deprotected by reaction with a suitable acid in a suitable solvent under suitable conditions to produce a reaction mass containing [R—(R*,R*)]-2-(4-fluorophenyl)-[β],[δ]-dihydroxy-5-(1-methylethyl)-3-phenyl-4-(phenylcarbamoyl)-1H-pyrrole-1-heptanoic acid tert-butyl ester (atorvastatin tert-butyl ester) of formula III followed by usual work up such as washings, evaporations etc., and then dissolving or extracting the resulting crude atorvastatin tert-butyl ester in the water immiscible solvent at a temperature of below about reflux temperature of the solvent used, more preferably at about 20° C. to about 110° C., and still more preferably at about 25° C. to about 80° C.

The solution obtained in step-(a) may optionally be subjected to carbon treatment. The carbon treatment can be carried out by methods known in the art, for example by stirring the solution with finely powdered carbon at a temperature of below about 70° C. for at least 15 minutes, preferably at a temperature of about 40° C. to about 70° C. for at least 30 minutes; and filtering the resulting mixture through hyflo to obtain a filtrate containing the atorvastatin tert-butyl ester by removing charcoal. Preferably, finely powdered carbon is an active carbon.

The solution obtained in step-(a) or step-(b) is optionally stirred at a temperature of about 20° C. to the reflux temperature of the solvent used for at least 20 minutes, and preferably at a temperature of about 25° C. to the reflux temperature of the solvent used from about 30 minutes to about 4 hours.

Removal of solvent in step-(c) is accomplished by, for example, substantially complete evaporation of the solvent, concentrating the solution and filtering the solid under inert atmosphere. Alternatively, the solvent may also be removed by evaporation. Evaporation can be achieved at sub-zero temperatures by the lyophilization or freeze-drying technique. The solution may also be completely evaporated in, for example, a pilot plant Rota vapor, a Vacuum Paddle Dryer or in a conventional reactor under vacuum above about 720 mm Hg by flash evaporation techniques by using an agitated thin film dryer (“ATFD”), or evaporated by spray drying to obtain a dry amorphous powder.

One of the preferred methodologies to remove the solvent involves spray-drying, in which a solution of atorvastatin tert-butyl ester is sprayed into the spray drier at the flow rate ranging from 10 to 300 ml/hr, preferably flow rate is 40 to 200 ml/hr. The air inlet temperature to the spray drier used may range from 25 to 150° C., preferably from 60° C. to 110° C. and the outlet air temperature used may range from 30 to 90° C. Another preferred method is vertical agitated thin-film drying (or evaporation). Agitated thin film evaporation technology involves separating the volatile component using indirect heat transfer coupled with mechanical agitation of the flowing film under controlled condition.

The distillation process can be performed at atmospheric pressure or reduced pressure. Preferably the solvent is removed at a pressure of about 760 mm Hg or less, more preferably at about 400 mm Hg or less, still more preferably at about 80 mm Hg or less, and most preferably from about 30 to about 80 mm Hg.

The substantially pure amorphous form of atorvastatin tert-butyl ester obtained by the above processes may be further dried in, for example, Vacuum Tray Dryer, Rotocon Vacuum Dryer, Vacuum Paddle Dryer or pilot plant Rota vapor, to further lower residual solvents.

Atorvastatin and its pharmaceutically acceptable salts thereof can be prepared in high purity by using the substantially pure amorphous atorvastatin tert-butyl ester obtained by the methods disclosed herein, by known methods. Preferable pharmaceutically acceptable salts of atorvastatin include alkali and alkaline earth metal salts such as lithium, sodium, potassium, magnesium, calcium, strontium, and more preferably sodium, magnesium and calcium.

According to another aspect of the present invention, there is provided pharmaceutical compositions comprising amorphous atorvastatin calcium prepared according to processes of the present invention in any of its embodiments, and one or more pharmaceutically acceptable excipients.

According to another aspect of the present invention, there is provided a process for preparing a pharmaceutical formulation comprising combining amorphous atorvastatin calcium prepared according to processes of the present invention in any of its embodiments, with one or more pharmaceutically acceptable excipients.

According to another aspect of the present invention, there is provided pharmaceutical compositions comprising atorvastatin calcium polymorphic Form A1 and one or more pharmaceutically acceptable excipients.

According to another aspect of the present invention, there is provided pharmaceutical compositions comprising atorvastatin calcium polymorphic Form A1 prepared according to processes of the present invention in any of its embodiments and one or more pharmaceutically acceptable excipients.

According to another aspect of the present invention, there is provided a process for preparing a pharmaceutical formulation comprising combining atorvastatin calcium polymorphic Form A1 prepared according to processes of the present invention in any of its embodiments, with one or more pharmaceutically acceptable excipients.

Yet another embodiment of the present invention is directed to pharmaceutical compositions comprising at least a therapeutically effective amount of substantially pure atorvastatin calcium polymorphic Form A1 of the present invention. Such pharmaceutical compositions may be administered to a mammalian patient in any dosage form, e.g., liquid, powder, elixir, injectable solution, etc. Dosage forms may be adapted for administration to the patient by oral, buccal, parenteral, ophthalmic, rectal and transdermal routes or any other acceptable route of administration. Oral dosage forms include, but are not limited to, tablets, pills, capsules, troches, sachets, suspensions, powders, lozenges, elixirs and the like. The atorvastatin calcium polymorphic Form A1 of the present invention may also be administered as suppositories, ophthalmic ointments and suspensions, and parenteral suspensions, which are administered by other routes. The dosage forms may contain the atorvastatin calcium polymorphic Form A1 of the present invention as is or, alternatively, may contain the atorvastatin calcium polymorphic Form A1 of the present invention as part of a composition. The pharmaceutical compositions may further contain one or more pharmaceutically acceptable excipients. Suitable excipients and the amounts to use may be readily determined by the formulation scientist based upon experience and consideration of standard procedures and reference works in the field, e.g., the buffering agents, sweetening agents, binders, diluents, fillers, lubricants, wetting agents and disintegrants described hereinabove.

Capsule dosages will contain the atorvastatin calcium polymorphic Form A1 of the present invention within a capsule which may be coated with gelatin. Tablets and powders may also be coated with an enteric coating. The enteric-coated powder forms may have coatings containing at least phthalic acid cellulose acetate, hydroxypropylmethyl cellulose phthalate, polyvinyl alcohol phthalate, carboxy methyl ethyl cellulose, a copolymer of styrene and maleic acid, a copolymer of methacrylic acid and methyl methacrylate, and like materials, and if desired, they may be employed with suitable plasticizers and/or extending agents. A coated capsule or tablet may have a coating on the surface thereof or may be a capsule or tablet comprising a powder or granules with an enteric-coating.

Tableting compositions may have few or many components depending upon the tableting method used, the release rate desired and other factors. For example, the compositions of the present invention may contain diluents such as cellulose-derived materials like powdered cellulose, microcrystalline cellulose, microfine cellulose, methyl cellulose, ethyl cellulose, hydroxyethyl cellulose, hydroxypropyl cellulose, hydroxypropylmethyl cellulose, carboxymethyl cellulose salts and other substituted and unsubstituted celluloses; starch; pregelatinized starch; inorganic diluents such calcium carbonate and calcium diphosphate and other diluents known to one of ordinary skill in the art. Yet other suitable diluents include waxes, sugars (e.g. lactose) and sugar alcohols like mannitol and sorbitol, acrylate polymers and copolymers, as well as pectin, dextrin and gelatin.

Other excipients contemplated by the present invention include binders, such as acacia gum, pregelatinized starch, sodium alginate, glucose and other binders used in wet and dry granulation and direct compression tableting processes; disintegrants such as sodium starch glycolate, crospovidone, low-substituted hydroxypropyl cellulose and others; lubricants like magnesium and calcium stearate and sodium stearyl fumarate; flavorings; sweeteners; preservatives; pharmaceutically acceptable dyes and glidants such as silicon dioxide.

The following examples are provided to enable one skilled in the art to practice the invention and are merely illustrate the process of this invention. However, it is not intended in any way to limit the scope of the present invention.

### EXAMPLES

### Example 1

### Preparation of (4R-cis)-6-[2-[3-phenyl-4-(phenylcarbamoyl)-2-(4-fluorophenyl)-5-(1-methylethyl)-pyrrol-1-yl]-ethyl]-2,2-dimethyl-[1,3]-dioxane-4-yl-acetic acid tertiary butyl ester (dimethyl ketal of atorvastatin tert-butyl ester) of formula IV

A mixture of (±)-4-fluoro-α-(2-methyl-1-oxopropyl)-γ-oxo-N,β-diphenyl benzenebutane-amide (300 g), (4R-cis)-1,1-dimethylethyl 6-(2-aminoethyl)-2,2-dimethyl-1,3-dioxane-4-acetate (206 g), cyclohexane (2100 ml), toluene (450 ml), tetrahydrofuran (450 ml) and trimethyl acetic acid (48.6 g) were heated to reflux temperature 80 to 85° C. for about 50 to 55 hours. The reaction mass was cooled and diluted with toluene (600 ml). The reaction mixture was then washed initially with water (2×1500 ml), then with 5% aqueous sodium carbonate solution (1200 ml) followed by water (1500 ml) and brine (20%, 1500 ml). The organic layer was charcoalized with activated carbon (15 g) for 1 hour at 25 to 30° C. followed by filtration to remove charcoal from solution through hyflo bed and washed bed with toluene (300 ml). The filtrate was concentrated under vacuum and solvent exchange was performed using isopropyl alcohol (600 ml) and the reaction mass was concentrated. Isopropyl alcohol (1500 ml) was added to the concentrated mass and heated at 80 to 85° C. to dissolve the reaction mass in isopropyl alcohol. The resulting solution was cooled gradually to 20 to 25° C. (approximately 2 to 3 hours) and stirred at this temperature for 10 to 12 hours. Next, the mass was cooled to 0 to 5° C. and stirred for 3 hours at this temperature. The resulted solid was filtered and washed with chilled isopropyl alcohol and dried at 40 to 45° C. to produce 300 g of pure (4R-cis)-6-[2-[3-phenyl-4-(phenylcarbamoyl)-2-(4-fluorophenyl)-5-(1-methylethyl)-pyrrol-1-yl]-ethyl]-2,2-dimethyl-[1,3]-dioxane-4-yl-acetic acid tert-butyl ester (HPLC purity: 99.30%).

### Example 2

### Preparation of amorphous atorvastatin tertiary butyl ester of formula III

(4R-Cis)-6-[2-[3-phenyl-4-(phenylcarbamoyl)-2-(4-fluorophenyl)-5-(1-methylethyl)-pyrrol-1-yl]-ethyl]-2,2-dimethyl-[1,3]-dioxane-4-yl-acetic acid tert-butyl ester (10 g) was suspended in isopropanol (150 ml) under stirring. This was followed by the addition of 1N hydrochloric acid (21 ml) at 25 to 30° C. The reaction mixture was heated at 40 to 45° C. and stirred for 4 to 5 hours at 40 to 45° C. The resulted mass was cooled at 20 to 25° C. The pH of reaction mass was adjusted to 7 to 7.5 by adding 5% w/v sodium bicarbonate solution maintaining temperature between 25 to 30° C. The neutralized mass was concentrated under vacuum at 40 to 45° C. Next, water (100 ml) and dichloromethane (100 ml) were added to the concentrated mass and stirred the reaction mass for 10 minutes. The organic layer was separated off and the aqueous layer was extracted with dichloromethane (2×100 ml).The combined organic layer was washed with water (200 ml) followed by 30% sodium chloride solution (200 ml). The resulting organic layer was distilled off under vacuum at 35 to 40° C. till dryness to yield 8 g of amorphous atorvastatin tertiary butyl ester.

### Example 3

### Preparation of Form A1 of atorvastatin calcium (atorvastatin hemi-calcium salt)

Atorvastatin sodium (100.0 g) was dissolved in purified water (7000 ml) under stirring at 40 to 45° C. The clear solution was cooled at 25 to 30° C. The pH of solution was adjusted about 7.9 to 8.5 by adding 6N hydrochloric acid at 25 to 30° C. under stirring. Next, 10% aqueous calcium acetate hemihydrate solution (17.6 g in 170 ml purified water) was added slowly at 25 to 30° C. The reaction mass was stirred for 10 to 12 hours at 25 to 30° C. The resulted solid was filtered and washed with purified water and dried at 55 to 60° C. under vacuum to produce 70.6 g of Form A1 of atorvastatin calcium.

### Example 4

### Preparation of Form A1 of atorvastatin calcium

Atorvastatin sodium (100 g) was dissolved in purified water (7000 ml) under stirring at 40 to 45° C. The resulted clear solution was cooled at 25 to 30° C. Next, 10% aqueous calcium acetate hemi hydrate solution (17.6 g in 170 ml purified water) was added slowly at 25 to 30° C. The resulted mass was stirred for 10 to 12 hours at 25 to 30° C. The resulting solid was filtered, washed with purified water and the dried at 55 to 60° C. under vacuum to give 70 g of Form A1 of atorvastatin calcium.

### Example 5

### Preparation of Form A1 of atorvastatin calcium

Atorvastatin sodium (50 g) was dissolved in purified water (3500 ml) under stirring at 40 to 45° C. The clear solution was cooled at 25 to 30° C. The pH of solution was adjusted at about 8.0 to 8.5 by adding 6N hydrochloric acid at 25 to 30° C. under stirring. Next, amorphous atorvastatin calcium (5 g) was added at 25 to 30° C. The resulting mass was stirred for 15 minutes at 25 to 30° C. The 10% aqueous calcium acetate hemi hydrate solution (9.0 g in 90 ml purified water) was dumped at once maintaining temperature 25 to 30° C. The reaction mixture was stirred for 10 to 12 hours at 25 to 30° C. The resulting solid thus obtained was filtered, washed with purified water and then dried at 55 to 60° C. under vacuum to produce 36 g of Form A1 of atorvastatin calcium.

### Example 6

### Preparation of Form A1 of atorvastatin calcium

Atorvastatin sodium (50 g) was dissolved in purified water (3500 ml) under stirring at 40 to 45° C. The clear solution was cooled at 25 to 30° C. Next, amorphous atorvastatin calcium (5 g) was added at 25 to 30° C. The resulting mass was stirred for 15 minutes at 25 to 30° C. Next, 10% aqueous calcium acetate hemi hydrate solution (9.0 g in 90 ml purified water) was added slowly maintaining temperature at 25 to 30° C. The mass was stirred for 10 to 12 hours at 25 to 30° C. The resulted solid was filtered, washed with purified water and dried at 55 to 60° C. under vacuum to produce 39.8 g of Form A1 of atorvastatin hemi-calcium.

### Example 7

### Preparation of amorphous atorvastatin calcium salt

Atorvastatin calcium Form A1 (3.5 g) was suspended in acetone (6.5 g) under stirring followed by heating at 50 to 55° C. for 20 minutes to obtain clear solution. The resulting solution was filtered through hyflo bed and washed bed with acetone (1 g). The clear solution was concentrated to dryness in a Buchi Rotavapor apparatus under a vacuum. 2.80 g of amorphous atorvastatin calcium was obtained.

### Example 8

### Preparation of amorphous atorvastatin calcium salt

Crude amorphous atorvastatin hemi calcium (30.0 g) was suspended in methyl ethyl ketone (70.0 g) under stirring followed by heating at 50 to 55° C. for 20 minutes to obtain clear solution. The resulting solution was filtered through hyflo bed and washed bed with methyl ethyl ketone (15 g). The resulted clear solution was concentrated to dryness using laboratory spray dryer (Jay Instruments & Systems Pvt. Ltd. India, Model-LSD-48 mini Spray Dryer). 25.0 g of amorphous atorvastatin calcium was obtained.

### Example 9

### Preparation of amorphous atorvastatin calcium salt

Crude amorphous atorvastatin hemi calcium (35.0 g) was suspended in methyl ethyl ketone (65 g) under stirring followed by heating at 50 to 55° C. for 20 minutes to obtain clear solution. The resulting solution was filtered through hyflo bed and washed bed with methyl ethyl ketone (20 g). The resulted clear solution was concentrated to dryness using laboratory spray dryer (Jay Instruments & Systems Pvt. Ltd. India, Model-LSD-48 mini Spray Dryer). 29.0 g of amorphous atorvastatin calcium was obtained.

### Example 10

### Preparation of amorphous atorvastatin calcium salt

Atorvastatin hemi-calcium Form A1 (3.5 g) was suspended in dichloromethane (6.5 g) under stirring followed by heating at 40 to 45° C. for 20 minutes to obtain clear solution. The resulting solution was filtered through Hyflo bed and washed bed with dichloromethane (1.0 g). The clear solution was concentrated to dryness in a Buchi Rotavapor apparatus under a vacuum. 3.0 g of amorphous atorvastatin calcium was obtained.

### Example 11

### Preparation of amorphous atorvastatin calcium salt

Atorvastatin calcium Form A1 (5.0 g) was suspended in dichloromethane (14.30 g) under stirring followed by heating at 40 to 45° C. for 20 minutes to obtain clear solution. The resulting solution was filtered through Hyflo bed and washed bed with dichloromethane (2.0 g). The resulted clear solution was concentrated to dryness using laboratory spray dryer (Jay Instruments & Systems Pvt. Ltd. India, Model-LSD-48 mini Spray Dryer). 2.5 g of amorphous atorvastatin calcium was obtained.

1. A polymorphic Form A1 of atorvastatin calcium characterized by at least one, or more, of the following properties:
i) a powder X-ray diffraction pattern substantially in accordance with FIG. 1;
ii) a powder X-ray diffraction pattern having peaks at about 5.3, 8.3 and 15.7±0.2 degrees 2-theta;
iii) an IR spectrum substantially in accordance with FIG. 2;
iv) an IR spectrum having characteristic absorption bands at about 1661, 820 and 807 cm⁻¹; and
v) a DSC thermogram having an endotherm peak at about 164-167° C. substantially in accordance with FIG. 3.

2. A process for the preparation of atorvastatin calcium Form A1 of claim 1, which comprises:
a) providing a solution of an atorvastatin salt in water, wherein the salt of atorvastatin is an alkali metal salt, an alkaline-earth metal salt, an ammonium salt, an alkyl ammonium salt, an aryl ammonium salt, or an alkaryl ammonium salt;
b) optionally, adjusting the pH of the solution to about 7 to 9 with an acid, wherein the acid is an organic acid, an inorganic acid, or an aqueous solution of inorganic acid;
c) optionally, seeding the solution obtained in step-(a) or step-(b) with amorphous atorvastatin calcium;
d) combining the atorvastatin salt solution with a calcium salt, wherein the calcium salt is selected from the group consisting of calcium acetate, calcium propionate, calcium butyrate, calcium tartrate, calcium benzoate, calcium phthalate, calcium stearate, calcium dodecanoate, calcium ascorbate, calcium succinate, calcium methane sulfonate, calcium benzene sulfonate, calcium p-toluene sulfonate, CaCl₂, CaF₂, CaBr₂, CaI₂, calcium borate (B₄CaO₇), calcium tetrafluoroborate (CaBF₄), calcium carbonate (CaCO₃), monobasic calcium phosphate (Ca(H₂PO₄)₂), dibasic calcium phosphate (CaHPO4), tribasic calcium phosphate (Ca(PO₄)₂), calcium sulfate (CaSO₄) and calcium hydroxide (Ca(OH)₂), and hydrates thereof; and
e) isolating polymorphic Form A1 of atorvastatin calcium from the reaction mass by forcible or spontaneous crystallization, wherein the forcible crystallization is initiated by cooling, seeding, partial removal of the solvent from the solution, or a combination thereof.

3. The process of claim 2, wherein the atorvastatin salt used in step-(a) is atorvastatin sodium; and wherein the calcium salt used in step-(d) is calcium acetate.

4. (canceled)

5. (canceled)

6. The process of claim 2, wherein the solution in step-(a) is provided either by dissolving atorvastatin salt in water at a temperature of below about 90° C.; or by hydrolyzing atorvastatin tert-butyl ester of formula III with a base in a solvent under suitable conditions to produce a reaction mass containing crude atorvastatin salt, subjecting the reaction mass to washings, evaporations or extractions, and dissolving the resulting atorvastatin salt in water under stirring at a temperature of below about 90° C.

7. The process of claim 6, wherein the atorvastatin salt is dissolved in water at a temperature of about 30° C. to about 80° C.; and wherein the base is selected from the group consisting of ammonia, sodium hydroxide, magnesium hydroxide, potassium hydroxide, lithium hydroxide, sodium carbonate, potassium carbonate and lithium carbonate.

8. (canceled)

9. (canceled)

10. The process of claim 6, wherein the atorvastatin tert-butyl ester of formula III used is an amorphous form of atorvastatin tert-butyl ester characterized by a powder XRD pattern showing no peaks substantially in accordance with FIG. 5; and wherein the amorphous atorvastatin tert-butyl ester is prepared by a process comprising:
a) providing a solution of atorvastatin tert-butyl ester in a suitable water immiscible solvent selected from the group consisting of aromatic hydrocarbons, cyclic ethers, halogenated organic solvents, and mixtures thereof;
b) optionally, filtering the solvent solution to remove any extraneous matter; and
c) substantially removing the solvent from the solution to afford amorphous form of atorvastatin tert-butyl ester, wherein the removal of the solvent is accomplished by complete evaporation of the solvent, spray drying, vacuum drying, lyophilization or freeze drying, or a combination thereof.

11-15. (canceled)

16. The process of claim 2, wherein the solution obtained in step-(a) is optionally subjected to carbon treatment; wherein the combining in step-(d) is carried out by adding the calcium salt to the atorvastatin salt solution or by adding the atorvastatin salt solution to the calcium salt; wherein the calcium salt in step-(d) is used in the form of a solid, a neat liquid, or a solution of calcium salt dissolved in water; wherein the crystallization in step-(e) is carried out by cooling the solution at a temperature of below 30° C.; and wherein the solid obtained in step-(e) is collected by filtration or centrifugation.

17-22. (canceled)

23. The process of claim 16, wherein the addition is carried out slowly at a temperature of below about 50° C. for at least 15 minutes; wherein the reaction mass obtained after completion of addition process is stirred at a temperature of about 25° C. to about 50° C. from about 2 hours to 12 hours; and wherein the crystallization is carried out by cooling the solution at a temperature of about 0° C. to about 25° C.

24-35. (canceled)

36. A process for the preparation of amorphous form of atorvastatin calcium, characterized by a powder XRD pattern showing no peaks substantially in accordance with FIG. 4, which comprises:
a) providing a solution of atorvastatin calcium polymorphic Form A1 in a suitable solvent selected from the group comprising consisting of halogenated hydrocarbons, ketones, and mixtures thereof;
b) optionally, filtering the solvent solution to remove any extraneous matter; and
c) substantially removing the solvent from the solution to afford amorphous form of atorvastatin calcium, wherein the removal of the solvent is accomplished by complete evaporation of the solvent, spray drying, vacuum drying, lyophilization or freeze drying, or a combination thereof.

37. The process of claim 36, wherein the halogenated hydrocarbon solvent is selected from the group consisting of dichloromethane, 1,2-dichloroethane, chloroform, carbon tetrachloride, and mixtures thereof; wherein the ketone solvent is selected from the group consisting of acetone, methyl ethyl ketone, methyl isobutyl ketone, methyl tert-butyl ketone, and mixtures thereof; wherein the ratio of atorvastatin calcium salt to the halogenated hydrocarbon is about 0.5 g/mL; and wherein the ratio of atorvastatin calcium salt to the ketone solvent is about 26 to 39% w/w.

38-40. (canceled)

41. The process of claim 36, wherein the solution in step-(a) is provided by dissolving atorvastatin calcium polymorphic Form A1 in the solvent or by suspending the atorvastatin calcium polymorphic Form A1 in the solvent followed by heating the suspension to form a clear solution; wherein the dissolution is carried out at a temperature of about 25° C. to about 100° C.; wherein the solution obtained in step-(a) is optionally subjected to carbon treatment; wherein the solution obtained in step-(a) or step-(b) is optionally stirred at a temperature of about 30° C. to the reflux temperature of the solvent used for at least 20 minutes; and wherein the amorphous atorvastatin calcium obtained in step-(c) has a total purity of greater than about 99.9% as measured by HPLC.

42-52. (canceled)

53. The process of claim 10, wherein the water immiscible solvent used in step-(a) is selected from the group consisting of toluene, xylene, tetrahydrofuran, 1,4-dioxan, hexahydropyran, dichloromethane, 1,-2-dichloroethane, chloroform, carbon tetrachloride, and mixtures thereof.

54. (canceled)

55. The process of claim 10, wherein the solution in step-(a) is provided either by dissolving atorvastatin tert-butyl ester in the water immiscible solvent at a temperature of below about reflux temperature of the solvent used; or by a process comprising condensing (4R-cis)-1,1-dimethylethyl 6-(2-aminoethyl)-2,2-dimethyl-1,3-dioxane-4-acetate with (±)-4-fluoro-α-(2-methyl-1-oxopropyl)-γ-oxo-N,β-diphenylbenzene butaneamide under acidic conditions in a suitable solvent or a mixture of suitable solvents to produce (4R-cis)-6-[2-[3-phenyl-4-(phenylcarbamoyl)-2-(4-fluorophenyl)-5-(1-methylethyl)-pyrrol-1-yl]-ethyl]-2,2-dimethyl-[1,3]-dioxane-4-yl-acetic acid tert-butyl ester of formula IV, deprotecting the compound of formula IV by reaction with a suitable acid in a suitable solvent under suitable conditions to produce a reaction mass containing [R—(R*,R*)]-2-(4-fluorophenyl)-[β],[δ]-dihydroxy-5-(1-methylethyl)-3-phenyl-4-(phenylcarbamoyl)-1H-pyrrole-1-heptanoic acid tert-butyl ester (atorvastatin tert-butyl ester) of formula III, subjecting the reaction mass to washings, evaporations, and then dissolving or extracting the resulting crude atorvastatin tert-butyl ester in the water immiscible solvent at a temperature of below about reflux temperature of the solvent used; and wherein the dissolution or extraction is carried out at a temperature of about 25° C. to about 80° C.

56-58. (canceled)

59. The process of claim 10, wherein the solution obtained in step-(a) is optionally subjected to carbon treatment; wherein the solution obtained in step-(a) or step-(b) is optionally stirred at a temperature of about 20° C. to the reflux temperature of the solvent used for at least 20 minutes; and wherein the amorphous atorvastatin tert-butyl ester obtained has a total purity of greater than about 98%, or greater than about 99%, as measured by HPLC.

60-64. (canceled)

65. A pharmaceutical composition comprising a solid state form of atorvastatin calcium and one or more pharmaceutically acceptable excipients, wherein the solid state form of atorvastatin calcium is the atorvastatin calcium polymorphic form A1 of claim 1 or the amorphous atorvastatin calcium obtained as per the process of claim 36; and wherein the pharmaceutical composition is prepared by a process comprising combining the solid state form of atorvastatin calcium with one or more pharmaceutically acceptable excipients.

66-68. (canceled)

69. The pharmaceutical composition of claim 65, wherein the pharmaceutical composition is a solid dosage form, an oral suspension, a liquid, a powder, an elixir or an injectable solution.

70. (canceled)

71. The pharmaceutical composition of claim 65, wherein the atorvastatin calcium polymorphic form A1 has a particle size of less than or equal to about 400 microns.

72. The pharmaceutical composition of claim 71, wherein the atorvastatin calcium polymorphic form A1 has a D₉₀ particle size of less than or equal to about 300 microns, less than or equal to about 100 microns, less than or equal to about 60 microns, or less than or equal to about 15 microns.

73-75. (canceled)
